(19) United States
(12) Patent Application Publication
(10) Pub. No.: US 2020/0263161 A1
(43) Pub. Date: Aug. 20, 2020

(54) IMMOBILIZED ENZYME PRODUCTION LINE, METHOD OF MAKING AN IMMOBILIZED ENZYME PRODUCTION LINE, AND METHOD OF MAKING COMPOUNDS USING THE IMMOBILIZED ENZYME PRODUCTION LINE

(71) Applicant: Immortazyme Company Ltd., Calgary (CA)

(72) Inventor: Vincent Yacyshyn, Calgary (CA)

(21) Appl. No.: 16/785,698

(22) Filed: Feb. 10, 2020

Related U.S. Application Data
(60) Provisional application No. 62/807,782, filed on Feb. 20, 2019.

(51) Int. Cl. C12N 11/18; C12P 19/02; C11B 3/00

(52) U.S. Cl. CPC C12N 11/18; C11B 3/003; C12P 19/02

(57) ABSTRACT

An enzyme production line having a plurality of enzymes 3 bound to a support 4 for running a series of catalyzed reactions to convert a substrate 30 to a final product 32. A method of using the enzyme production line to form a final product 32 in which a substrate 30 contacts a first enzyme 3 bound to a support 4 to form an intermediate and contacting the intermediate with a second enzyme 3 bound to a support 4 to form a final product 32.

### FIELD OF THE INVENTION

The invention relates to a method of sculpting immobilized enzymes in a three-dimensional manner and optimizing the physicochemical interaction between the enzyme(s) and substrate to provide an enzyme production line, and a method of producing desired products using the enzyme production line.

### BACKGROUND OF THE INVENTION

Enzyme immobilization has traditionally been used for enhancement in enzyme productivity and increasing shelf life. Enzyme immobilization provides an excellent base for increasing availability of the enzyme to the substrate with greater turnover over a considerable period of time. Several natural and synthetic supports have been used. Immobilized enzymes are preferred over their free counterparts due to prolonged availability that curtails downstream redundancy and purification processes. Datta S. et al. Biotech. 2013 February; 3(1):1-9. Enzyme immobilization is the confinement of an enzyme to a phase (matrix/support) different for the one for the substrates and products. An ideal matrix must be: inert, have physical strength, stability, regenerability, ability to increase specific enzyme activity, reduce product inhibition, decrease nonspecific absorption, and prevent microbial contamination. Factors influencing immobilized enzyme performance include: hydrophobic partition, microenvironment of the carrier, multipoint attachment of the carrier, spacer or arm placement, diffusion constraints, presence of substrates or inhibitors, physical post-treatments, binding modes, physical pore size, and physical nature of the carrier. Techniques used for immobilization include: adsorption, covalent binding, affinity binding, and entrapment. Materials used for supports include: alginates, chitosans, collagen, carrageenan, gelatin, cellulose, starch, pectin, sepharose, synthetic polymers, zeolites, ceramics, celite, silica, glass, activated carbon, or charcoal. Datta S. et al. Biotech 2013. February; 3(1):1-9.

Immobilization of sequential enzyme has been reported using surface-tethered glycolytic enzymes. Mukai C. et al. Chem Biol. 2009 Sep. 25: 16(9): 1013-1020. The development of a complex hybrid inorganic-organic device for sequential enzyme performance faces several challenges including: the production of energy, the reported study uses recombinant hexokinase type 1 and glucose-6-phosphate isomerase capable of oriented immobilization on a nickel nitrilotriacetic acid modified surface. These enzymes showed sequential activity when tethered together. This was the first demonstration of using surface-tethered pathway components of sequential enzymes while tethered in series to a single, solid support. The pathway demonstrated the metabolism of glucose to pyruvate releasing energy that may be used to produce adenosine tricophosphate (ATP).

Methods have been reported for the immobilization of pectinases by sequential layering on chitosan beads. Ay S. S. et al. International Journal of Scientific and Technological Research Vol. 4, No. 6 (2018). This method used glutaraldehyde as an active agent for covalent binding. Increasing the density of the pectinases with multilayered binding (up to three layers) increased the density of the pectinases on the chitosan support but decreased the catalytic ability of the enzymes due to either restriction to the protein backbone or due to substrate accessibility limitations.

Similarly, three immobilized enzymes acting in series have been placed in a layer-by-layer assembly. This “layer-by-layer” deposition was made possible with the a biospecific complexation between Concanavalin A and sugar residues in the glycoenzymes. The layer-by-layer deposition allowed for a high ordinate architecture with high loading of the enzymes and functionality of the enzymes so that they may act in a series of catalyzed cascading reactions. Palazzo G. et al. Sensors and Actuators Vol. 202, 31 Oct. 2014, pager 217-223.

Sequential immobilization of urease to glycidyl methacrylate grafted to sodium alginate has also been accomplished enabling reactions to be catalyzed with different enzymes in an arranged order as a mutli-enzyme system. Akkaya A. et al. Journal of Molecular Catalysis. Vol 67, Issues 3-4. December 2010. Pages 195-201.

Sarah Baker et al at the Lawrence Livermore National Laboratory published a report in Nature Communications using a particulate methane monooxygenase to create a biocatalytic polymer material that converts methane to methanol with an embedded material in a silicone lattice to create a mechanically robust, gas-permeable membrane based on direct printing of micron-scale structures with controlled geometry. (Blanchette C. D. et al. Printable enzyme-embedded materials for methane to methanol conversion. Nat Commun. 2016; 7: 11900.)

U.S. Pat. No. 7,312,056 describes a method for “Enhancement of Enzyme Activity through Purification and Immobilization”. This patent describes an improved method for making an immobilized enzyme by treating an immobilization support with an aqueous solution comprising a cross-linking agent and polymeric aldehyde species with an active center species to produce a modified support, isolating this support, and treating an enzyme solution with the modified support to produce an immobilized enzyme. This art is enhanced with U.S. Pat. No. 7,892,805 entitled “Method of Enhancing Enzyme Activity and Enzyme Solution having Enhanced Activity”. This patent describes a method for treating a raw enzyme with purifying agents (carbon) to provide for the subsequent enhancement of the enzyme solution. Although silent to the subsequent immobilization processes, the combination of patents optimizes the enzyme prior to immobilization with a preferably carbon or silicon-based material.

The prior art is silent with regards to the methods necessary for sculpting three dimensional enzymes. Akin to U.S. Pat. No. 3,005,282, which describes toy building blocks that may be connected together by means of projections, extending from the faces of the elements and arranged so as the engage protruding portions of the adjacent element when two such elements are assembled. The prior art is silent to the use of such elements which may be printed as an enzyme substrate in a three-dimensional manner, coated with immobilized enzymes, and sculpted in such a manner to optimize the enzyme substrate interaction for previously non-feasible reactions.

U.S. Pat. No. 10,071,912 describes the use of carbon nano-materials. Networks of carbon (or silica) may be printed and used as the immobilization material. The carbon wall structures may be as small as 0.2 mg per cm cubed or lower. The carbon structures may be tubular, rod-like, or in the form of a web which have varying thicknesses and form a three-dimensional network structure and may ultimately be constructed in the manner of a sponge.

U.S. Pat. No. 8,818,737 describes “Methods, systems, algorhythms and means for describing the possible conformations of actual and theoretical proteins and for evaluating actual and theoretical proteins with respect to folding, overall shape and structural motifs”. Torsion angles and pitch motifs create a plurality of 27 vectors.

Complex reactions requiring multiple different enzyme steps are expensive, time consuming and are unpredictable.

Enzyme cofactors are now well-known. For example a cofactor can be a non-protein chemical compound or metallic ion that is required for an enzyme's activity. Cofactors can be considered “helper molecules” that assist in biochemical transformations.

### SUMMARY OF THE INVENTION

The present invention provides a plurality of different enzymes bound to at least one support in particular positions so that a product of a first bound enzyme is the precursor of a second bound enzyme and so on to form an enzyme production line so that a desired compound can be produced continuously by feeding a precursor into the enzyme production line. Cofactors can be used to selectively control the activity of each bound enzyme in the enzyme product line. A preferred cofactor is adenosine diphosphate (ADP).

The immobilized enzymes have the advantages of providing a desired order of enzyme action in a multi-step enzyme reaction, precise control over each step in the enzyme production line, enzyme stability, enzyme longevity, enhanced enzyme activity, and cost-effective benefits. Sculpting a plurality of immobilized enzymes in specific three-dimensional structures also provides additional advantages including enhanced physicochemical properties of the substrate and enzyme with regards to temperature, pressure, flow, volume, resistance, and physical proximity of the enzyme-substrate complex.

### BRIEF DESCRIPTION OF THE DRAWINGS

FIG. 1A (top view) and FIG. 1B (side view) illustrates an example of an enzyme reaction station 2 comprising an immobilized enzyme 3 bound to a hexagon shaped support 4.

FIG. 1C illustrates a close up of an immobilized enzyme 3 bound to a square support 4.

FIG. 2 illustrates a plurality of the enzyme reaction stations of FIGS. 1A and 1B of similar size forming a multi-step enzyme production line having a cylindrical shape.

FIG. 3 illustrates a plurality of different sized enzyme reaction stations of FIGS. 1A and 1B forming a multi-step production line having a conical shape.

### DETAILED DESCRIPTION OF THE INVENTION

The invention will be explained with reference to the attached non-limiting Figs.

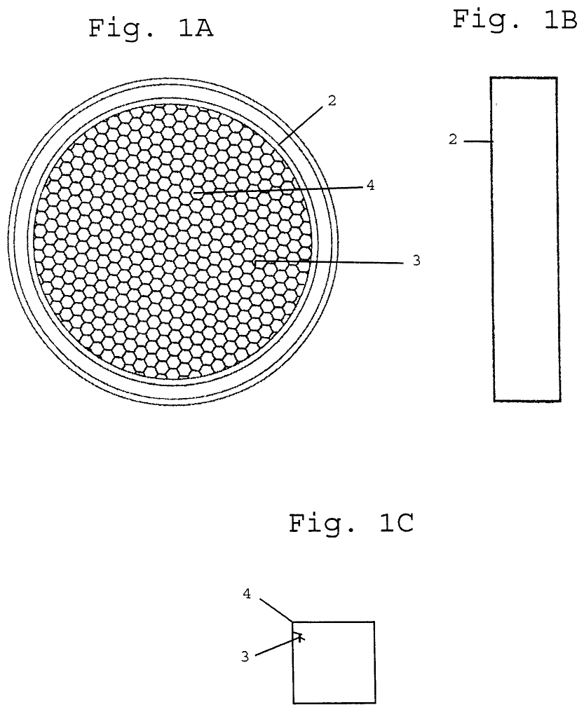

An embodiment of the invention includes an enzyme reaction station 2 comprising a support 4 having a first enzyme 3 bound to the support 4 as shown in FIGS. 1A-1C. The dimensions of the support 4 can be as desired and variable according to the necessary use. FIG. 1A is a top view of an enzyme reaction station 2 having a “honey comb” support 4 comprising carbon or silica, not limited thereto, and FIG. 1B is a side view of the enzyme reaction station 2. The enzyme reaction station 2 can have multiple different enzymes 3 bound on the same support 4, or only one type of bound enzyme 3 as desired. The term bound means that the enzyme 3 remains bonded to the support 4 during the reactions with the substrate 30 and intermediates to form the final product 32 and that the enzyme 3 exhibits enzyme properties while bound to the support 5.

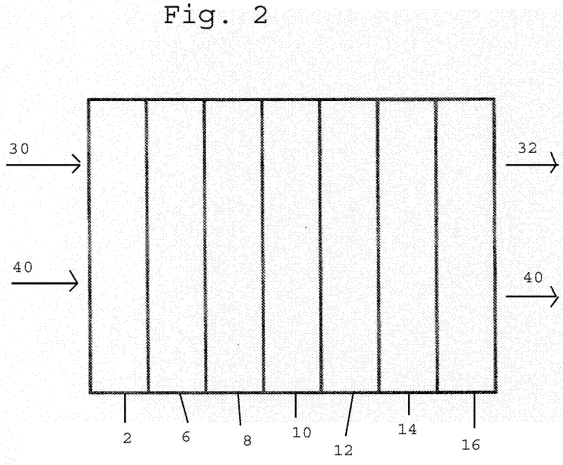

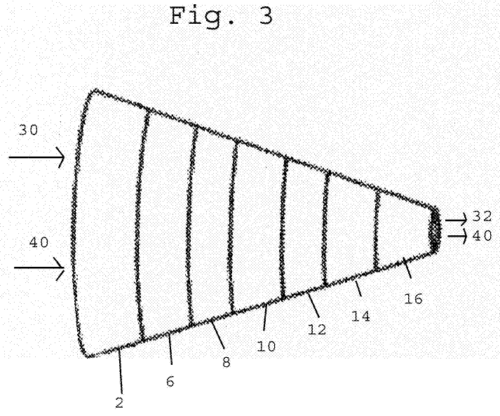

Multiple enzyme reaction stations 2 can be connected in any desired manner or order to form an enzyme production line as shown in FIGS. 2 and 3. Preferably, each enzyme reaction station 2 is customizable, based on the desired catalyzed reaction to be performed, with various components added or subtracted.

Support 4 is preferably porous as shown in FIG. 1A so that a substrate 30 and optionally a cofactor 40 can flow through the support 4 and contact the enzyme 3 bound to the support 4. Alternatively, the support 4 can be configured as desired so the substrate 30 and optional cofactor 40 can contact the enzyme 3 bound to the support 4. Any desired support 4 material can be utilized, including for example, alginates, chitosans, collagen, carrageenan, gelatin, cellulose, starch, pectin, sepharose, synthetic polymers, zeolites, ceramics, celite, silica, glass, activated carbon, or charcoal. The support 4 can have any desired shape, for example circles, triangles, squares, pentagons, hexagons, or any other shape. The flow paths through the support 4 can be as desired, for example, linear, curved, serpintine, or other.

Immobilization of an enzyme 3 on a support 4 is known. However, multiple enzymes bound at specific locations on a support and enzyme reaction stations are not known. Furthermore, it is not a simple matter to provide multiple enzymes bound at specific locations on a support. The present invention can utilize a plurality of immobilized enzymes and three-dimensional sculpting utilizing gas-to-liquid technology to provide sequential reactions below atmospheric pressures.

The support 4 should be configured to allow the flowing substrate 30 and optional cofactor 40 contact the bound enzyme 3. The enzyme reaction station 2 can be configured to contain the substrate 30 and optional cofactor 40 or the enzyme reaction station 2 can be inserted into a vessel for containing the substrate 30 and optional cofactor. The enzyme production line can be configured so that a first cofactor contacts a first bound enzyme, and a second cofactor contacts a second bound enzyme, and so on for additional bound enzymes.

FIGS. 2 and 3 illustrate examples of enzyme production lines comprising a plurality of enzyme reaction stations 2, 6, 8, 10, 12, 14 and 16. The substrate 30 and cofactor 40 flow into the first enzyme reaction station 2 wherein the first bound enzyme in the first enzyme reaction station 2 causes the substrate 30 to undergo a first catalyzed reaction to form a first intermediate. The first intermediate flows into the second enzyme reaction station 6 wherein the second bound enzyme in the second enzyme reaction station 6 causes the first intermediate to undergo a second catalyzed reaction and form a second intermediate. The second intermediate flows into the third enzyme reaction station 8 wherein the third bound enzyme in the third enzyme reaction station 8 causes the second intermediate to undergo a third catalyzed reaction and form a third intermediate. The third intermediate flows into the forth enzyme reaction station 10 wherein the fourth bound enzyme in the fourth enzyme reaction station 10 causes the third intermediate to undergo a fourth catalyzed reaction and form a fourth intermediate. The fourth intermediate flows into the fifth enzyme reaction station 12 wherein the fifth bound enzyme in the fifth enzyme reaction station 12 causes the fourth intermediate to undergo a fifth catalyzed reaction and form a fifth intermediate. The fifth intermediate flows into the sixth enzyme reaction station 14 wherein the sixth bound enzyme in the sixth enzyme reaction station 14 causes the fifth intermediate to undergo a sixth catalyzed reaction and form a sixth intermediate. The sixth intermediate flows into the seventh enzyme reaction station 16 wherein the seventh bound enzyme in the seventh enzyme reaction station 16 causes the sixth intermediate to undergo a seventh catalyzed reaction and form a final product 32 exiting the enzyme production line.

Final product 32 and cofactor 40 flow from the enzyme production line. Any number of enzyme reaction stations 2, 6, 8, 10, 12, 14 and 16 can be utilized. The product can be altered as desired, for example, by changing the number of enzyme reaction stations, the order of the enzyme reaction stations, the type of enzyme(s) in each enzyme reaction station, and/or by altering the reaction conditions in each separate enzyme reaction station, for example, temperature, pH, flow rate, adjuvants, cofactors, electromagnetic energy, humidity, or any other, and combinations thereof

The present invention is not limited by the following Examples. The enzyme production line can have different shapes, enzymes, and sizes. The enzyme production line can be formed by different methods.

Example 1—Carbon dioxide may be captured enzymatically. Carbonic anhydrase enzymes have been known to accelerate the hydration of neutral aqueous CO2 molecules to ionic bicarbonate species. (Alain C. Pierre Chemical Engineering Vol. 2012. Article ID 753687. 22 pages.)

Types of carbonic anhydrase available to catalyze CO2 include the following subgroups-cytosolic, mitochondrial, secreted, or membrane-binding. These enzymes work in two different mechanisms by “hydrase” and subsequently “esterase” mechanisms. Carbonic anhydrase has been immobilized as scrubbers (on a solid material in a packed bed reactor—CO2 Solutions) or on thin aqueous films with dissolved carbonic anhydrase. The core of this liquid membrane is a 330 micrometer thick enzyme solution in an aqueous phosphate buffer squeezed in between two microporous hydrophobic polypropylene membranes retained by thin metal grids to ensure the liquid membrane thickness and rigidity. This liquid-gas film method has been used by the National Aeronautics and Space Administration (NASA). The limitations with this method include drying of the enzyme with prolonged use.

Different strategies for the accelerated CO2 absorption in packed columns by the application of the biocatalyst carbonic anhydrase has been studied by Leimbrink, M. et al energy Procedia 114(2017)781-794. The use of aqueous N-Methyl diethanlamine solution and the enzyme carbonic anhydrase has been used in a packed column pilot plant.

The present invention allows for the solid-liquid location of carbonic anhydrase activity to be expanded into a gas-liquid mechanism. The Fischer-Tropsch process may be used by immobilizing the enzyme on a three dimensional carbon structure, assembling this structure as components so that the immobilized enzyme may interact with a CO2 concentrated gas to optimize the enzyme substrate conditions and allow for solid-gas interaction.

Carbonic anhydrase was immobilized by use of U.S. Pat. No. 7,312,056. A “skeleton” of carbon or silicon is three dimensionally printed as a building block tailored to the CO2 gas stream. The building block subsequently has the carbonic anhydrase immobilized on it. This process is repeated until the complete three dimensional structure is sculpted or completed in such a manner so as to optimize the interaction of the gas-liquid interaction of CO2 and carbonic anhydrase according to the method of Fischer-Tropsch.

By “sculpting” the enzyme as shown in FIG. 1.A. the surface area, temperature, pressure, and humidity of the reaction conditions between carbon dioxide and carbonic anhydrase may be optimized. Heretofore, this has not been possible without three-dimensional enzyme “sculpting”. Either a cylinder (Figure2.) or a cone (FIG. 3.) may be used to further optimize the enzymatic reaction as it proceeds.

Example 2—Conversion of CO2 into glucose using human waste is a requirement for prolonged space travel. Solid waste includes H2O (75%) and biomass (25%). Liquid waste includes: H2O (91-96%), urea, uric acid, and creatinine. The Martian atmosphere includes: CO2 (95.97%), argon (1.93%), nitrogen (1.89%), oxygen (0.146%), carbon monoxide (0.0557%) with pressure at 0.087 psi, 600 Pascals. The creation of either pyruvate, oxaloacetate, and glycerol may all be intermediate candidates for subsequent enzymatic conversion to glucose using enzyme immobilization, assembly of the three dimensional, sculpted immobilized structures into a sequential apparatus for the conversion of pyruvate, oxaloacetate, or glycerol to glucose. Glycerol may be converted to dihydroxyacetone phosphate and subsequently enzymatically converted to using triose phosphate isomerase to glyceraldehyde 3-phosphate or pyruvate may be converted to oxaloacetate by pyruvate carboxylase to oxaloacetate which may be converted enzymatically by phosphoenolpyruvate carboxylase to phosphoenolpyruvate which may be converted enzymatically by enolase to 2-phosphoglycerate which may be converted enzymatically by phosphoglycerate mutase to 3-phosphoglycerate which may be converted enzymatically by phosphoglycerate kinase to 1,3-bisphosphoglycerate which may be converted enzymatically by glyceraldehyde 3-phosphate dehydrogenase to glyceraldehyde 3-phosphate. The glyceraldehyde 3-phosphate may be converted enzymatically by aldolase to fructose 1,6-bisphosphate which may be enzymatically converted by fructose 1,6-bisphophatase to fructose 6-phosphate which may be converted enzymatically by phosphoglucose isomerase to glucose 6-phosphate which may be converted enzymatically by glucose 6-phosphatase to glucose.

The enzymes noted above may be immobilized according to the method of U.S. Pat. No. 7,312,056. The enzymes are affixed to a three dimensional structure. The sequential enzymes may be modelled or “sculpted” such that each step in the complex biochemical reaction is optimized.

The feasibility of performing the listed complex biochemical reaction can be facilitated by the technological advances outlined for immobilization of the respectively required enzymes, interlocking these three dimensional structures in a sculpted manner to optimize the interaction between the enzyme and substrate such that the final products can be produced.

This example illustrates sequential enzymatic reactions that may be possible in zero gravity. By placing the enzymes in a solid state material and sculpting them sequentially in a cylinder form as shown in FIG. 2., the apparatus may be sealed and the reaction able to proceed. A feat not possible with present technology.

Example 3—Heavy or viscous oil may be biochemically refined by altering the viscosity of the oil using various lipases immobilized to a three-dimensional structure as shown in FIG. 1.

The uniqueness of the present invention includes, but is not limited to:
1) Previously identified silicate or carbon immobilization processes may be modified or controlled in a new way to now affix enzyme on a novel three dimensional structure.
2) The three dimensional structure can be assembled in a component manner allowing for optimization of enzyme immobilization and assembly of complex geometric structures.
3) The complex three dimensional structure created in 2) can be customized to optimize the interaction between the enzyme and substrate complexes.
4) The complex forms of 2) may be assembled in a sequential manner with different enzymes such that complex chemical reactions previously impossible, can now be accomplished.

While the claimed invention has been described in detail and with reference to specific embodiments thereof, it will be apparent to one of ordinary skill in the art that various changes and modifications can be made to the claimed invention without departing from the spirit and scope thereof.

1. An enzyme production line comprising:
a support;
a first enzyme bound to a first location of the support; and
a second enzyme bound to a second location of the support, wherein the enzyme production line is configured so that a first precursor contacts the first enzyme to form a first intermediate, and the first intermediate contacts the second enzyme to form a product.

2. The enzyme production line according to claim 1, further comprising a source of a cofactor to selectively control the activity at least one of the first or second enzyme.

3. The enzyme production line according to claim 2, wherein the cofactor comprises adenosine diphosphate (ADP).

4. The enzyme production line according to claim 1, further comprising a third enzyme bound to a third location of the support, wherein the enzyme production line is configured so that a first precursor contacts the first enzyme to form a first intermediate, the first intermediate contacts the second enzyme to form a second intermediate, and the second intermediate contacts the third enzyme to form the product.

5. The enzyme production line according to claim 4, further comprising a fourth enzyme bound to a fourth location of the support, wherein the enzyme production line is configured so that a first precursor contacts the first enzyme to form a first intermediate, the first intermediate contacts the second enzyme to form a second intermediate, the second intermediate contacts the third enzyme to form a third intermediate, and the third intermediate contacts the fourth enzyme to form the product.

6. The enzyme production line according to claim 1, wherein the support is porous.

7. An enzyme production line comprising a plurality of enzyme reaction stations in communication, each enzyme reaction station comprising an enzyme bound to a support, wherein the enzyme exhibits enzyme properties while bound to the support.

8. The enzyme production line according to claim 7, further comprising a source of cofactor connected to the enzyme reaction stations.

9. The enzyme production line according to claim 7, wherein
a first enzyme reaction station comprising a first support and a first enzyme bound to the support; and
a second enzyme reaction station comprising a second support and a second enzyme bound to the second support, wherein the first enzyme reaction station is in communication with the second enzyme reaction chamber.

10. The enzyme production line according to claim 7, wherein the plurality of enzyme stations are removably connected to change an order a precursor flows through the enzyme stations.

11. The enzyme production line according to claim 7, wherein the plurality of enzyme stations are removably connected to change a number of enzyme stations.

12. A method of making a product using an enzyme production line comprising:
providing an enzyme production line comprising a support, a first enzyme bound to a first location of the support, and a second enzyme bond to a second location of the support;
contacting a precursor with the first enzyme and a first cofactor to form a first product; and
contacting the first product with the second enzyme and a second cofactor to form a second product.

13. The method according to claim 12, further comprising altering a product produce by altering reaction conditions in at least one of the enzyme reaction stations.

14. The method according to claim 13, wherein the reaction condition comprises at least one of temperature, pH, flow rate, adjuvants, cofactors, electromagnetic energy, humidity, or combinations thereof.

15. A method of producing a product using an enzyme production line comprising:
providing enzyme production line comprising a plurality of connected enzyme reaction stations, each enzyme reaction station comprising a support and an enzyme bound to the support;
adding a substrate into a first enzyme reaction station and allowing a first reaction to occur catalyzed by the first enzyme to form a first product; and
adding the first product to the second enzyme reaction station and allowing a second reaction to occur catalyzed by the second enzyme to form a second product.

16. The method according to claim 15, further comprising altering a product produce by changing the number of enzyme reaction stations.

17. The method according to claim 15, further comprising altering a product produce by changing a type of enzyme reaction station.

18. The method according to claim 15, further comprising altering a product produce by changing an order of the enzyme reaction stations.

19. The method according to claim 15, further comprising altering a product produce by altering reaction conditions in at least one of the enzyme reaction stations.

20. The method according to claim 15, wherein the reaction condition comprises at least one of temperature, pH, flow rate, adjuvants, cofactors, electromagnetic energy, humidity, or combinations thereof.
